(19) United States
(12) Patent Application Publication
(10) Pub. No.: US 2026/0183253 A1
(43) Pub. Date: Jul. 2, 2026

(54) TREATING LIVER CANCERS WITH HYDROXYUREAMETHYL ACYLFULVENE

(71) Applicant: Lantern Pharma Inc., Dallas, TX (US)

(72) Inventors: Jianli Zhou, Dallas, TX (US); Aditya Kulkarni, Dallas, TX (US); Kishor Bhatia, Atlanta, GA (US)

(73) Assignee: Lantern Pharma Inc., Dallas, TX (US)

(21) Appl. No.: 19/546,266

(22) Filed: Feb. 20, 2026

Related U.S. Application Data
(63) Continuation of application No. PCT/US24/43699, filed on Aug. 23, 2024.
(60) Provisional application No. 63/534,270, filed on Aug. 23, 2023.

(51) Int. Cl. A61K 31/17; A61K 31/44; A61K 31/513; A61K 31/704; A61K 33/243; A61K 45/06; A61P 35/00

(52) U.S. Cl. CPC A61K 31/17; A61K 31/44; A61K 31/513; A61K 31/704; A61K 33/243; A61K 45/06; A61P 35/00

(57) ABSTRACT

A method for treating liver cancer, particularly in subjects exhibiting homologous recombination deficiency (HRD) positivity, includes using hydroxyureamethyl acylfulvene. The compound can be administered as a monotherapy or in combination with other chemotherapeutic agents and/or radiotherapy.

### CROSS-REFERENCE TO RELATED APPLICATIONS

This is application a continuation of International Patent Application No PCT/US24/43699, filed on Aug. 23, 2024, which claims priority to and the benefit of U.S. Provisional Patent Application Ser. No. 63/534,270, filed on Aug. 23, 2023, each of which is hereby incorporate by reference in its entirety.

### TECHNICAL FIELD

This application relates to liver cancer treatments and more specifically this application relates to cancer treatments using an acylfulvene.

### BACKGROUND

Liver cancer, medically referred to as hepatocellular carcinoma (HCC), originates in the liver cells, also known as hepatocytes. As one of the most prevalent types of cancer globally, HCC poses a significant health challenge, particularly in regions with high incidences of chronic liver conditions. These underlying conditions often include cirrhosis, hepatitis B, or hepatitis C infections, which are major risk factors for the development of liver cancer. Due to its typically asymptomatic nature in the early stages, liver cancer is frequently diagnosed at more advanced stages, contributing to its high mortality rate.

The clinical presentation of liver cancer can be varied and often overlaps with symptoms of other liver-related disorders. Common symptoms include persistent abdominal pain or discomfort, particularly in the upper right quadrant, swelling or a noticeable mass in the abdomen, and unexplained weight loss. Patients may also experience a reduced appetite, extreme fatigue, and symptoms of jaundice, such as yellowing of the skin and eyes. Additionally, liver cancer can lead to the accumulation of fluid in the abdomen, known as ascites, further contributing to discomfort and swelling.

Treatment strategies for liver cancer are highly individualized and depend on several critical factors, including the stage and extent of the cancer, the patient's overall health, liver function, and the presence of underlying liver disease. Surgical options, such as resection to remove the tumor or liver transplantation, are considered for patients with localized cancer and good liver function. Non-surgical treatments include radiation therapy to target and destroy cancer cells, chemotherapy to slow the progression of the disease, and targeted drug therapies that specifically attack cancerous cells while sparing healthy tissue. In many cases, a multidisciplinary approach combining these treatments may offer the best chance of disease control and prolonging survival.

The prognosis for individuals diagnosed with liver cancer is influenced by several factors, including the stage at which the cancer is detected, the patient's overall health, and how well the cancer responds to treatment. Generally, early detection and intervention are associated with more favorable outcomes. However, liver cancer is often diagnosed at an advanced stage, where treatment options become more limited, and the prognosis is poorer. Preventative measures, including regular medical check-ups, vaccination against hepatitis B, managing hepatitis C infections, maintaining a healthy lifestyle, and reducing alcohol intake, play a crucial role in mitigating the risk of developing liver cancer.

Despite advances in medical research and treatment, liver cancer remains a formidable challenge. There is a continuous need for the development of more effective and innovative therapies to improve survival rates and the quality of life for those affected by this aggressive disease.

Accordingly, there is always a need for improved treatments for liver cancer.

### SUMMARY

The application discloses a method for treating liver cancer, particularly in subjects exhibiting homologous recombination deficiency (HRD) positivity, using hydroxyureamethyl acylfulvene. The compound can be administered as a monotherapy or in combination with other chemotherapeutic agents and/or radiotherapy. Various administration routes and dosages are disclosed herein. The application also highlights the use of hydroxyureamethyl acylfulvene as an adjuvant or neoadjuvant therapy in conjunction with surgical resection of liver tumors. Furthermore, the identification of specific biomarkers, such as deficiencies in DNA repair pathways like NER, can be utilized to predict the efficacy of the treatment. The application encompasses hydroxyureamethyl acylfulvene and includes additional therapeutic strategies, including combination with immune checkpoint inhibitors.

One aspect includes a method in which the liver cancer is hepatocellular carcinoma.

Another aspect includes a method in which the hydroxyureamethyl acylfulvene is administered as monotherapy.

Another aspect includes a method in which the hydroxyureamethyl acylfulvene is administered in combination with one or more chemotherapeutic agents selected from the group consisting of sorafenib, doxorubicin, cisplatin, and 5-fluorouracil.

Another aspect includes a method in which hydroxyureamethyl acylfulvene is administered in conjunction with radiotherapy.

Another aspect includes a method in which the hydroxyureamethyl acylfulvene has the following structure:

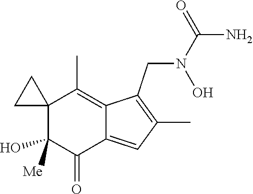 [SMILES: CC1=C(CN(O)C(N)=O)C2=C(C)C3(CC3)[C@@](C)(O)C(=O)C2=C1]

### BRIEF DESCRIPTION OF THE DRAWING

FIG. 1 illustrates the IC50 (nM) data, demonstrating that LP-184 exhibits nanomolar potency against various liver cancer cell lines.

### DETAILED DESCRIPTION

This application provides a therapy for treating liver cancers. In some embodiments, the therapy involves administering agents such as acylfulvene or illudin. In other embodiments, the therapy involves administering a combination of therapies. Additionally, the therapy can be used to treat both the biochemical occurrence and recurrence of liver cancer, where an acylfulvene (e.g., hydroxyureamethyl acylfulvene) or a salt thereof is administered in a therapeutically effective amount to the subject.

### Illudin or Acylfulvene

In one embodiment, this application includes the use of an illudin or illudin analog (e.g., acylfulvene). Acylfulvene is a class of cytotoxic semi-synthetic derivatives of illudin, a natural product that can be extracted from the jack o'lantern mushroom (Omphalotus olearius). Acylfulvene, derived from the sesquiterpene illudin S by treatment with acid (reverse Prins reaction), is far less reactive to thiols than illudin S.

In one example, the acylfulvene is (−)-hydroxyureamethyl acylfulvene (termed LP-184 by Lantern Pharma Inc.), which shifts light negatively, is shown below:

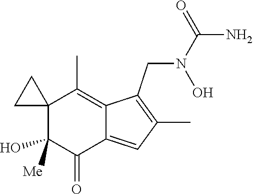 [SMILES: CC1=C(CN(O)C(N)=O)C2=C(C)C3(CC3)[C@@](C)(O)C(=O)C2=C1]

In another example, the acylfulvene is (+)-hydroxyureamethyl acylfulvene (termed LP-284 by Lantern Pharma Inc), which shifts light positively, is shown below:

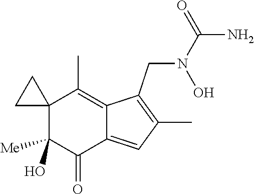 [SMILES: CC1=C(CN(O)C(N)=O)C2=C(C)C3(CC3)[C@](C)(O)C(=O)C2=C1]

(+)—hydroxyureamethyl acylfulvene and (−)—hydroxyureamethyl acylfulvene are enantiomers and are now known publicly.

In another example, the acylfulvene is Irofulven.

### Nucleotide Excision Repair (NER) Pathway

Nucleotide Excision Repair (NER) is a DNA repair mechanism that plays a crucial role in maintaining the integrity of the DNA molecule in liver cancer cells by identifying and repairing various types of damage, particularly bulky lesions that distort the DNA helix. These lesions, which can contribute to the development and progression of liver cancer, are often caused by environmental factors such as UV radiation, chemicals, and other mutagens.

NER is a versatile pathway that can repair a wide range of DNA lesions, making it crucial for the maintenance of genomic stability. Deficiencies in the NER pathway can lead to a higher susceptibility to cancer. Studying NER has also provided insights into various disease processes.

NER positivity can be determined through various methods, including genetic testing, genomic profiling, or specific biomarker assays. These tests aim to identify genetic or genomic alterations associated with NER, such as mutations or loss of function in genes involved in the homologous recombination pathway.

### Homologous Recombination Deficiency (HRD) Positive

Homologous recombination deficiency (HRD) positivity refers to the presence of genetic or genomic alterations in a liver tumor that indicate a defect or impairment in the homologous recombination DNA repair pathway. HRD positivity is often assessed in the context of liver cancer, particularly in relation to predicting the response to certain treatments.

When liver tumors exhibit HRD, they are more likely to have difficulties repairing DNA damage, such as double-stranded breaks. This can make them more susceptible to certain therapies that exploit these repair deficiencies.

HRD positivity in liver cancer can be determined through various methods, including genetic testing, genomic profiling, or specific biomarker assays. These tests aim to identify genetic or genomic alterations associated with HRD, such as mutations or loss of function in genes involved in the homologous recombination pathway.

Assessing HRD positivity in liver cancer has significant implications for treatment decisions. For example, in liver cancer, HRD positivity may serve as a predictive biomarker for the response to specific drugs. These drugs block alternative DNA repair pathways relied upon by HRD-positive liver cancer cells, leading to their selective targeting and cell death.

In one embodiment, acylfulvene or hydroxyureamethyl acylfulvene or its salt may be administered either prior to, concurrently with, or subsequent to the administration of another agent.

One embodiment includes a method of treating liver cancer in a subject in need thereof.

Another embodiment provides a method for treating hepatocellular carcinoma (HCC) by administering hydroxyureamethyl acylfulvene as a monotherapy. This method involves the administration of a therapeutically effective dose of hydroxyureamethyl acylfulvene to

patients diagnosed with HCC. The compound can be administered via oral, intravenous, or intraperitoneal routes, with the dosage adjusted based on the patient's weight, age, and disease severity. The treatment regimen may involve daily, weekly, or bi-weekly administration, depending on the desired therapeutic effect and patient tolerance.

Another embodiment provides a method for treating liver cancer in which hydroxyureamethyl acylfulvene is used in combination with one or more conventional chemotherapeutic agents to enhance the treatment of liver cancer. Suitable chemotherapeutic agents include, but are not limited to, sorafenib, doxorubicin, cisplatin, and 5-fluorouracil.

Another embodiment provides a method for treating liver cancer in which hydroxyureamethyl acylfulvene is employed as an adjuvant therapy following surgical resection of liver tumors. After the primary tumor has been surgically removed, patients are administered hydroxyureamethyl acylfulvene to target residual cancer cells and prevent recurrence.

Another embodiment provides a method for treating liver cancer in which hydroxyureamethyl acylfulvene is encapsulated in nanoparticles for targeted delivery to liver cancer cells. This approach utilizes nanoparticles that are designed to preferentially accumulate in liver tissues, particularly in cancerous cells, thereby increasing the local concentration of the drug while reducing systemic exposure and toxicity.

Another embodiment provides a method for treating liver cancer in which hydroxyureamethyl acylfulvene is used in conjunction with radiotherapy. The compound may be administered before, during, or after radiation treatment to sensitize liver cancer cells to radiation, thereby enhancing the effectiveness of radiotherapy.

Another embodiment includes methods for preventing liver cancer in an individual at risk of having liver cancer, comprising the step of administering to the individual an effective amount of hydroxyureamethyl acylfulvene. In some embodiments, the individual has a liver condition selected from the group consisting of hepatitis B or C, cirrhosis of the liver, benign liver tumors, hemangiomas, hepatic adenomas, and focal nodular hyperplasia. In some embodiments, the method further comprises administering at least one additional therapeutic agent. Examples of additional therapeutic agents include growth inhibitory agents, such as cytotoxic agents, peptides, small molecules, and antibodies.

Another embodiment includes a pharmaceutical composition comprising a therapeutically effective amount of an illudin, an illudin analog, or a pharmaceutically acceptable salt thereof. The illudin analog can be hydroxyureamethyl acylfulvene.

In another embodiment, the second therapeutic is one or more chemotherapeutic agents selected from camptothecin derivatives, paclitaxel, docetaxel, epothilone B, 5-FU, gemcitabine, oxaliplatin, cisplatin, carboplatin, melphalan, dacarbazine, temozolomide, doxorubicin, imatinib, erlotinib, bevacizumab, cetuximab, and a Raf kinase inhibitor.

In another embodiment, the second therapeutic is one or more chemotherapeutic agents selected from paclitaxel or cisplatin.

The term “combination therapy” includes the administration of the therapeutic agents as described above in further combination with other biologically active ingredients and nondrug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In another aspect, a composition or combination therapy herein, or a pharmaceutically acceptable salt or solvate thereof, may be administered in combination with radiation therapy. Radiation therapy can also be administered in combination with a composition of the present invention and another chemotherapeutic agent described herein as part of a multiple-agent therapy.

In another embodiment, methods are provided for treating or preventing a liver cell proliferative disorder associated with increased expression or activity of a protein having at least 70%, 80%, 85% 90%, 95%, 98% or 99% amino acid sequence identity to PTGR1 (human), comprising administering to an individual in need of such treatment an effective amount of hydroxyureamethyl acylfulvene, thereby effectively treating or preventing the liver cell proliferative disorder. In some embodiments, the cell proliferative disorder is liver cancer. In some embodiments, the individual has a liver condition selected from the group consisting of hepatitis B or C, cirrhosis of the liver, benign liver tumors, hemangiomas, hepatic adenomas, and focal nodular hyperplasia.

Combination therapy can be achieved by administering two or more agents, e g., an acylfulvene and one or more other therapeutic agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases, even longer intervals are possible. While in many cases it is desirable that the two or more agents used in combination therapy be present within the patient's body at the same time, this need not be so.

The methods of combination therapy may result in a synergistic effect, wherein the effect of a combination of compounds or other therapeutic agents is greater than the sum of the effects resulting from the administration of any of the compounds or other therapeutic agents as single agents. A synergistic effect may also be an effect that cannot be achieved by administration of any of the compounds or other therapeutic agents as single agents. The synergistic effect may include, but is not limited to, an effect of treating liver cancer by reducing tumor size, inhibiting tumor growth, or increasing the survival of the subject. The synergistic effect may also include reducing liver cancer cell viability, inducing liver cancer cell death, and inhibiting or delaying liver cancer cell growth.

Therapeutically effective doses can vary, as recognized by those skilled in the art, depending on the diseases treated, the severity of the disease, the route of administration, the age and general health condition of the patient, excipient usage, the possibility of co-usage with other therapeutic treatments such as the use of other agents, and the judgment of the treating physician. For example, guidance for selecting an effective dose can be determined by reference to the prescribing information for acylfulvene or hydroxyureamethyl acylfulvene or journal discussion of the same.

The term “effective amount” as used herein refers to the amount of an agent needed to alleviate at least one or more symptoms of liver cancer and relates to a sufficient amount of pharmacological composition to provide the desired effect. The term “therapeutically effective amount” therefore refers to an amount of the agent that is sufficient to provide a particular effect when administered to a typical subject. An effective amount may be an amount sufficient to decrease the symptoms of liver cancer. For liver cancer therapy, efficacy in vivo can, for example, be measured by assessing the duration of survival, time to disease progression (TTP), response rates (RR), duration of response, and/or quality of life. Effective amounts may vary, as recognized by those skilled in the art, depending on route of administration, excipient usage, and co-usage with other agents. An effective amount as used herein, in various contexts, would also include an amount sufficient to delay the development of a symptom of liver cancer, alter the course of a symptom (for example, but not limited to, slowing the progression of a symptom), or reverse a symptom. Thus, it is not generally practicable to specify an exact “effective amount.” However, for any given case, an appropriate “effective amount” can be determined by one of ordinary skill in the art using only routine experimentation.

The dosage ranges for the administration of an agent according to the methods described herein depend upon, for example, the form of the agent, its potency, and the extent to which symptoms, markers, or indicators of liver cancer are desired to be reduced, for example, the percentage reduction desired for tumor growth. The dosage should not be so large as to cause adverse side effects. Generally, the dosage will vary with the age, condition, and sex of the patient and can be determined by one of skill in the art. The dosage can also be adjusted by the individual physician in the event of any complication.

The term “therapeutically effective amount,” as used herein, refers to an amount of a pharmaceutical agent to treat, ameliorate, or prevent liver cancer or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is liver cancer. In another aspect, the disease or condition to be treated is a liver cell proliferative disorder.

The efficacy of an agent described herein in the treatment of liver cancer or to induce a response (e.g., in solid cancers or blood cancers) can be determined by the skilled clinician. However, a treatment is considered “effective treatment,” as the term is used herein, if one or more of the signs or symptoms of liver cancer are altered in a beneficial manner, other clinically accepted symptoms are improved or even ameliorated, or a desired response is induced, e.g., by at least 10% following treatment according to the methods described herein. Efficacy can be assessed, for example, by measuring a marker, indicator, symptom, and/or the incidence of liver cancer or any other measurable parameter appropriate, e.g., tumor size and/or growth rate. Efficacy can also be measured by a failure of an individual to worsen as assessed by hospitalization or need for medical interventions (i.e., progression of the disease is halted). Methods of measuring these indicators are known to those of skill in the art and/or are described herein. Treatment includes any treatment of liver cancer in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of liver cancer means that amount which, when administered to a subject in need thereof, is sufficient to result in effective treatment as that term is defined herein, for that disease. Efficacy of an agent can be determined by assessing physical indicators of liver cancer or a desired response. It is well within the ability of one skilled in the art to monitor efficacy of administration and/or treatment by measuring any one of such parameters, or any combination of parameters. Efficacy can be assessed in animal models of liver cancer, for example, treatment of liver cancer in a mouse model. When using an experimental animal model, efficacy of treatment is evidenced when a statistically significant change in a marker is observed, e.g., tumor size and/or growth rate. In some embodiments, the therapeutically effective amount of hydroxyureamethyl-acylfulvene, acylfulvene, Irofulven or a pharmaceutically acceptable salt thereof is selected from the group consisting of 0.5 mg/day, 1 mg/day, 2.5 mg/day, 5 mg/day, 10 mg/day, 20 mg/day, 30 mg/day, 60 mg/day, 90 mg/day, 120 mg/day, 150 mg/day, 180 mg/day, 210 mg/day, 240 mg/day, 270 mg/day, 300 mg/day, 360 mg/day, 400 mg/day, 440 mg/day, 480 mg/day, 520 mg/day, 580 mg/day, 600 mg/day, 620 mg/day, 640 mg/day, 680 mg/day, and 720 mg/day.

The administration dose should be adjusted based on the individual needs of the patient. For example, the administered dosage of hydroxyureamethyl-acylfulvene will be in

the range from about 0.05 mg/kg to about 30 mg/kg, preferably 1 mg/kg to 30 mg/kg; or 500 mg-3000 mg flat dose. Thus, one or more doses of about 0.5 mg/kg, 2.0 mg/kg, 4.0 mg/kg, 10 mg/kg, or 30 mg/kg or 500 mg-3000 mg flat dose (or any combination thereof) may be coadministered to the patient. The preferred dosage of acylfulvene will be in the range from 20 mg/kg to about 150 mg/kg, preferably 1 mg/kg to 10 mg/kg. An initial higher loading dose, followed by one or more lower doses, may also be administered. In some cases, it is more suitable to apply the lower end of the above-described dosage ranges, while in other cases, the higher dosages may be used without causing harmful side effects. The goal is often to give the maximum tolerated dosage.

The term “treat” includes both therapeutic treatment and prophylactic treatment (reducing the likelihood of development). Both terms mean to decrease, suppress, attenuate, diminish, arrest, or stabilize the development or progression of liver cancer, lessen the severity of the disease, or improve the symptoms associated with the disease.

The pharmaceutical compositions can be included in a container, pack, or dispenser together with instructions for administration.

The composition of the present invention is capable of forming salts. The composition can form more than one salt per molecule, e.g., mono-, di-, tri-. All of these forms are also contemplated within the scope of the claimed invention.

As used herein, “pharmaceutically acceptable salts” refer to derivatives of the compounds of the present invention wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethanesulfonic, acetic, ascorbic, benzenesulfonic, benzoic, bicarbonic, carbonic, citric, edetic, ethanedi sulfonic, 1,2-ethanesulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methanesulfonic, naphsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluenesulfonic, and the commonly occurring amine acids, e.g., glycine, alanine, phenylalanine, arginine, etc.

It should be understood that all references to pharmaceutically acceptable salts include solvent addition forms (solvates) of the same salt.

As used herein, the term “selectively” means tending to occur at a higher frequency in one population than in another population. The compared populations can be cell populations. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in liver cancer cells if it occurred greater than twice as frequently in liver cancer cells as compared to normal liver cells.

The composition, or pharmaceutically acceptable salts or solvates thereof, are administered orally, nasally, transdermally, pulmonarily, inhalationally, buccally, sublingually, intraperitoneally, subcutaneously, intramuscularly, intravenously, rectally, intrapleurally, intrathecally, and parenterally. In one embodiment, the compound is administered orally. One skilled in the art will recognize the advantages of certain routes of administration.

The dosage regimen utilizing the compounds is selected in accordance with a variety of factors including type, species, age, weight, sex, and medical condition of the patient; the severity of the condition to be treated; the route of administration; the liver and hepatic function of the patient; and the particular compound or salt thereof employed. An ordinarily skilled physician or veterinarian can readily determine and prescribe the effective amount of the drug required to prevent, counter, or arrest the progress of liver cancer.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure, the skilled artisan can identify and employ other components and methodology useful for practicing the present invention.

As used herein, a “subject in need thereof” is a subject having a precancerous condition. Preferably, a subject in need thereof has liver cancer. A “subject” includes a mammal. The mammal can be, e.g., any mammal, e.g., a human, primate, bird, mouse, rat, dog, cat, cow, horse, goat, camel, sheep, or pig. Preferably, the mammal is a human. The subject of the present invention includes any human subject who has been diagnosed with, has symptoms of, or is at risk of developing liver cancer or a precancerous condition.

A subject in need thereof may have refractory or resistant liver cancer. “Refractory or resistant liver cancer” means liver cancer that does not respond to treatment. The liver cancer may be resistant at the beginning of treatment, or it may become resistant during treatment. In some embodiments, the subject in need thereof has liver cancer recurrence following remission on the most recent therapy. In some embodiments, the subject in need thereof received and failed all known effective therapies for liver cancer treatment. In some embodiments, the subject in need thereof received at least one prior therapy. In certain embodiments, the prior therapy is monotherapy. In certain embodiments, the prior therapy is combination therapy.

In some embodiments, a subject in need thereof may have secondary liver cancer as a result of previous therapy. “Secondary liver cancer” means cancer that arises due to or as a result of previous carcinogenic therapies, such as chemotherapy.

Liver cancer is a group of diseases that may cause almost any sign or symptom. The signs and symptoms will depend on where the cancer is, the size of the cancer, and how much it affects nearby organs or structures. If liver cancer spreads (metastasizes), symptoms may appear in different parts of the body.

Treating liver cancer can result in a reduction in the size of a tumor. A reduction in the size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. The size of a tumor may be measured by any reproducible means of measurement. The size of a tumor may be measured as a diameter of the tumor.

Treating liver cancer results in a decrease in the number and size of tumors. Preferably, after treatment, the number or size of tumors is reduced by 5% or greater relative to the number prior to treatment; more preferably, the number or size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating liver cancer can result in a decrease in the number of metastatic lesions in other tissues or organs distant from the primary tumor site. Preferably, after treatment, the number of metastatic lesions is reduced by 5% or greater relative to the number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating liver cancer can result in an increase in the average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in the average survival time of a population may be measured by any reproducible means. An increase in the average survival time of a population may be measured, for example, by calculating for a population the average length of survival following initiation of treatment with an active compound. An increase in the average survival time of a population may also be measured, for example, by calculating for a population the average length of survival following completion of a first round of treatment with an active compound.

Treating liver cancer can result in an increase in the average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in the average survival time of a population may be measured by any reproducible means. An increase in the average survival time of a population may be measured, for example, by calculating for a population the average length of survival following initiation of treatment with an active compound. An increase in the average survival time of a population may also be measured, for example, by calculating for a population the average length of survival following completion of a first round of treatment with an active compound.

Treating liver cancer can result in an increase in the average survival time of a population of treated subjects in comparison to a population receiving monotherapy with a drug that is not a compound of the present invention, or a pharmaceutically acceptable salt or solvate thereof. Preferably, the average survival time is increased by more than 30 days; more preferably, by more than 60 days; more preferably, by more than 90 days; and most preferably, by more than 120 days. An increase in the average survival time of a population may be measured by any reproducible means. An increase in the average survival time of a population may be measured, for example, by calculating for a population the average length of survival following initiation of treatment with an active compound. An increase in the average survival time of a population may also be measured, for example, by calculating for a population the average length of survival following completion of a first round of treatment with an active compound.

Treating liver cancer can result in a decrease in the mortality rate of a population of treated subjects in comparison to a population receiving carriers alone. Treating liver cancer can result in a decrease in the mortality rate of a population of treated subjects in comparison to an untreated population. Treating liver cancer can result in a decrease in the mortality rate of a population of treated subjects in comparison to a population receiving monotherapy with a drug that is not a compound of the present invention, or a pharmaceutically acceptable salt or solvate thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating liver cancer can result in a decrease in tumor growth rate. Preferably, after treatment, the tumor growth rate is reduced by at least 5% relative to the number prior to treatment; more preferably, the tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating liver cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after prior tumor shrinkage that followed treatment. A decrease in tumor regrowth is indicated by the failure of tumors to recur after treatment has stopped.

Treating or preventing a liver cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a liver cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a liver cell proliferative disorder can result in a decrease in the size of an area or zone of cellular proliferation. Preferably, after treatment, the size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. The size of an area or zone of cellular proliferation may be measured by any reproducible means of measurement. The size of an area or zone of cellular proliferation may be measured as a diameter or width of an area or zone of cellular proliferation.

Treating or preventing a liver cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleomorphism.

Administering a composition of the present invention to a cell or a subject in need thereof can result in the modulation (i.e., stimulation or inhibition) of an activity of a protein methyltransferase of interest.

Treating liver cancer or a liver cell proliferative disorder can result in cell death, and preferably, cell death results in a decrease of at least 10% in the number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. The number of cells in a population may be measured by any reproducible means. The number of cells in a population can be measured by fluorescence-activated cell sorting (FACS), immunofluorescence microscopy, and light microscopy. Methods of measuring cell death are as shown in Li et al., Proc. Natl. Acad. Sci. USA. 100(5): 2674-8, 2003. In an aspect, cell death occurs by apoptosis.

Preferably, an effective amount of a composition of the present invention, or a pharmaceutically acceptable salt or solvate thereof, is not significantly cytotoxic to normal liver cells. A therapeutically effective amount of a compound is not significantly cytotoxic to normal liver cells if administration of the compound in a therapeutically effective amount does not induce cell death in greater than 10% of normal liver cells. A therapeutically effective amount of a compound does not significantly affect the viability of normal liver cells if administration of the compound in a therapeutically effective amount does not induce cell death in greater than 10% of normal liver cells. In an aspect, cell death occurs by apoptosis.

Contacting a cell with a composition of the present invention, or a pharmaceutically acceptable salt or solvate thereof, can induce or activate cell death selectively in liver cancer cells. Administering to a subject in need thereof a compound of the present invention, or a pharmaceutically acceptable salt or solvate thereof, can induce or activate cell death selectively in liver cancer cells. Contacting a cell with a composition of the present invention, or a pharmaceutically acceptable salt or solvate thereof, can induce cell death selectively in one or more cells affected by a liver cell proliferative disorder. Preferably, administering to a subject in need thereof a composition of the present invention, or a pharmaceutically acceptable salt or solvate thereof, induces cell death selectively in one or more cells affected by a liver cell proliferative disorder.

The present invention relates to a method of treating or preventing liver cancer by administering a composition of the present invention, or a pharmaceutically acceptable salt or solvate thereof, to a subject in need thereof, where administration of the composition of the present invention, or a pharmaceutically acceptable salt or solvate thereof, results in one or more of the following: prevention of liver cancer cell proliferation by accumulation of cells in one or more phases of the cell cycle (e.g., G1, G1/S, G2/M), or induction of cell senescence, or promotion of liver tumor cell differentiation; promotion of cell death in liver cancer cells via cytotoxicity, necrosis, or apoptosis, without a significant amount of cell death in normal liver cells, antitumor activity in animals with a therapeutic index of at least 2. As used herein, “therapeutic index” is the maximum tolerated dose divided by the efficacious dose.

The terms “liver cancer” and “cancerous” refer to or describe the physiological condition in mammals that is typically characterized by unregulated liver cell growth/proliferation. Examples of liver cancer include, but are not limited to, hepatocellular carcinoma, hepatoma, hepatoblastoma, cholangiocarcinoma, hepatic carcinoma, sarcoma, lymphoma, and hepatic angiosarcoma. Liver cancer also includes cancer that originated in the liver and has metastasized to another part of the body.

An “individual at risk of having liver cancer” refers to an individual having a higher-than-average propensity of acquiring liver cancer. Examples of individuals at risk of having liver cancer include, without limitation, individuals having hepatitis, e.g., hepatitis B or C, cirrhosis of the liver, benign liver tumors, hemangiomas, hepatic adenomas, and focal nodular hyperplasias.

The term “kit” means a combination partner as defined above can be dosed independently or by use of different fixed combinations with distinguished amounts of the combination partners, i.e., simultaneously or at different time points. The parts of the kit of parts can then, e.g., be administered simultaneously or chronologically staggered, that is, at different time points and with equal or different time intervals for any part of the kit of parts. The ratio of the total amounts of the combination partners to be administered in the combined preparation can be varied. The combination partners can be administered by the same route or by different routes.

The term “level of expression” or “expression level,” as used herein, refers to the amount of a polynucleotide, mRNA, or an amino acid product or protein in a biological sample.

One skilled in the art may refer to general reference texts for detailed descriptions of known techniques discussed herein or equivalent techniques. These texts can, of course, also be referred to in making or using an aspect of the invention.

### Examples

In order that the disclosure disclosed herein may be more efficiently understood, examples are provided below. It should be understood that these examples are for illustrative purposes only and are not to be construed as limiting the disclosure in any manner.

### Cell Culture and Treatment Protocol:

Liver cancer cell lines including SNU387, SNU475, SNU449, and HEPG2, were obtained from the American Type Culture Collection (ATCC), ensuring standardized and reliable cell sources. By treating the above liver cancer cell lines with varying concentrations of LP-184, the testing demonstrates how effective the drug is in inhibiting or killing cancer cells across different genetic backgrounds and phenotypes of hepatocellular carcinoma (HCC). Cells were seeded into 96-well plates at a density optimized for exponential growth, allowing sufficient space for cellular proliferation during the course of the experiment. Each well was seeded with an equal number of cells to ensure consistency across the experiment. Following seeding, cells were allowed to adhere and acclimatize for 24 hours at 37° C. in a humidified incubator with 5% CO2, maintaining optimal growth conditions. Once acclimated, the cells were treated with either a vehicle control (DMSO) or varying concentrations of LP-184. Treatment was carried out in triplicates to ensure statistical significance and reproducibility of the results. The drug was administered at different concentrations, ranging from sub-therapeutic to potentially cytotoxic levels, to establish a dose-response curve. The treated cells were incubated for 72 hours under the same controlled conditions (37° C., 5% CO2).

Following the 72-hour treatment period, cell viability was assessed using the CellTiter-Fluor® assay from Promega, a sensitive fluorescence-based assay that quantifies the number of viable cells based on the presence of active cellular proteases. To each well, 50 pl of GF-AFC reagent was added, consisting of 10 pl of GF-AFC substrate diluted in 5 ml of the assay buffer, prepared freshly for each plate to ensure reagent stability and consistency.

The contents of each well were mixed gently to distribute the reagent evenly across the cells.

The plates were then incubated for an additional 30 minutes at 37° C. to allow the enzymatic reaction to occur, resulting in the cleavage of the fluorogenic substrate by viable cells. This reaction produces a fluorescent signal, which is directly proportional to the number of live cells in each well. The fluorescence signal was measured using a plate reader set to an excitation wavelength of 400 nm and an emission wavelength of 505 nm, parameters optimized for the GF-AFC substrate.

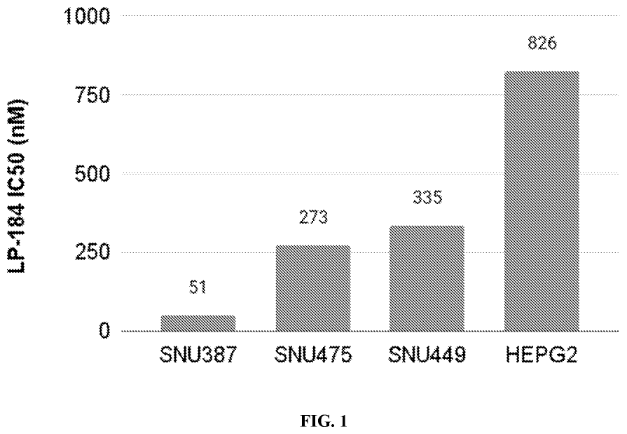

FIG. 1 illustrates the IC50 (nM) data, demonstrating that LP-184 exhibits nanomolar potency against various liver cancer cell lines. Fluorescence signals obtained from wells containing no cells (blank wells) were subtracted from all readings to eliminate background fluorescence and ensure accurate measurement of cell viability. The resulting baseline-adjusted relative fluorescence units (RFUs) were then normalized against the DMSO-treated control wells, which were set as 100% viability. This normalization allowed for the comparison of the effects of LP-184 across different concentrations.

The half-maximal inhibitory concentration (IC50) of LP-184 was calculated using GraphPad Prism, a software tool commonly employed for analyzing dose-response relationships. The IC50 value represents the concentration of LP-184 at which 50% of the cell population is inhibited or killed, providing a key indicator of the drug's potency. The IC50 values were derived from the dose-response curve generated by plotting the normalized RFUs against the logarithm of LP-184 concentrations, allowing for accurate determination of the LP-184's efficacy in treating or kill cancerous liver cells.

1. A method of treating liver cancer in a subject in need thereof, comprising administering to the subject a therapeutically effective amount of hydroxyureamethyl acylfulvene or a pharmaceutically acceptable salt thereof.

2. The method of claim 1, wherein the liver cancer is hepatocellular carcinoma (HCC).

3. The method of claim 1, wherein the liver cancer is cholangiocarcinoma.

4. The method of claim 1, wherein the hydroxyureamethyl acylfulvene is administered as monotherapy.

5. The method of claim 1, wherein the hydroxyureamethyl acylfulvene is administered in combination with one or more additional therapeutic agents for treating liver cancer.

6. The method of claim, wherein the one or more additional therapeutic agents comprise a chemotherapeutic agent.

7. The method of claim, wherein the one or more additional therapeutic agents comprise radiotherapy.

8. The method of claim 1, wherein the hydroxyureamethyl acylfulvene is administered by a route selected from the group consisting of oral, intravenous, intraperitoneal, subcutaneous, and intramuscular administration.

9. The method of claim 1, wherein the subject exhibits homologous recombination deficiency (HRD) positivity.

10. The method of claim, further comprising identifying one or more cancer cells in the subject that are deficient in homologous recombination-dependent DNA repair relative to normal liver cells.

11. The method of claim 9, wherein the subject's liver cancer exhibits nucleotide excision repair (NER) pathway deficiency.

12. The method of claim, wherein the presence of the NER pathway deficiency is used as a predictor of clinical response to the hydroxyureamethyl acylfulvene.

13. The method of claim, wherein the subject is selected as having increased expression or activity of PTGR1 in tumor tissue relative to non-tumor liver tissue.

14. The method of claim 1, wherein the hydroxyureamethyl acylfulvene is (−)-hydroxyureamethyl acylfulvene.

15. The method of claim 2, wherein the subject exhibits HRD positivity and the hydroxyureamethyl acylfulvene is administered intravenously.

16. The method of claim 1, wherein the hydroxyureamethyl acylfulvene is administered intravenously.

17. The method of claim, wherein the hydroxyureamethyl acylfulvene is administered using a liposomal delivery system.

18. The method of claim, wherein the hydroxyureamethyl acylfulvene is encapsulated in nanoparticles configured for targeted delivery to liver cancer cells.

19. The method of claim 1, wherein the hydroxyureamethyl acylfulvene is administered at a dosage of about 0.5 mg/kg once daily for 21 consecutive days.

20. The method of claim 1, wherein the hydroxyureamethyl acylfulvene is administered at a dosage from about 0.5 mg/kg to about 30 mg/kg.

21. The method of claim 1, wherein the hydroxyureamethyl acylfulvene is administered as adjuvant therapy following surgical resection of a liver tumor.

22. The method of claim 1, wherein the hydroxyureamethyl acylfulvene is administered as neoadjuvant therapy prior to surgical resection of a liver tumor.

23. The method of claim 6, wherein the chemotherapeutic agent is selected from sorafenib, doxorubicin, cisplatin, and 5-fluorouracil.

24. The method of claim 5, further comprising administering an immune checkpoint inhibitor.

25. The method of claim 1, wherein the subject has refractory or resistant liver cancer.

26. The method of claim 1, wherein the therapeutically effective amount induces cell death selectively in one or more liver cancer cells without inducing cell death in more than about 10% of normal liver cells.

27. The method of claim 1, wherein effectiveness of treatment is assessed by monitoring biomarkers associated with DNA repair deficiencies.

28. A method of treating hepatocellular carcinoma in a subject in need thereof, comprising administering to the subject hydroxyureamethyl acylfulvene or a pharmaceutically acceptable salt thereof, wherein (a) the subject exhibits homologous recombination deficiency (HRD) positivity; (b) the subject's liver cancer exhibits nucleotide excision repair (NER) pathway deficiency; (c) the subject is selected as having increased expression or activity of PTGR1 in tumor tissue relative to non-tumor liver tissue; and (d) the NER pathway deficiency is used as a predictor of clinical response to the hydroxyureamethyl acylfulvene.ne.

29. The method of claim 28, wherein the hydroxyureamethyl acylfulvene is administered using a liposomal delivery system.

30. The method of claim 28, wherein the hydroxyureamethyl acylfulvene is administered at a dosage of about 0.5 mg/kg once daily for 21 consecutive days.

31. The method of claim 28, wherein the hydroxyureamethyl acylfulvene is administered as adjuvant therapy following surgical resection of a liver tumor.

32. The method of claim 28, wherein the hydroxyureamethyl acylfulvene is administered as neoadjuvant therapy prior to surgical resection of a liver tumor.

33. The method of claim 28, further comprising administering an immune checkpoint inhibitor.

34. A kit comprising hydroxyureamethyl acylfulvene, or a pharmaceutically acceptable salt thereof, and instructions for administering the hydroxyureamethyl acylfulvene to a subject to treat liver cancer.

35. The kit of claim, wherein the instructions comprise instructions to administer the hydroxyureamethyl acylfulvene to a subject selected as exhibiting homologous recombination deficiency (HRD) positivity.

36. The kit of claim, further comprising reagents for detecting one or more biomarkers indicative of HRD positivity and/or NER pathway deficiency in a biological sample obtained from the subject.

37. The kit of claim, further comprising reagents for detecting one or more biomarkers indicative of HRD positivity or NER pathway deficiency in a biological sample obtained from the subject.
